SEQUENCE LISTING (70 sequences)

SEQ ID NO: 1 - DNA, 16 bp, synthetic construct
  source 1..16: mol_type = other DNA
cgccgagcagctgtct

SEQ ID NO: 2 - DNA, 18 bp, synthetic construct
  source 1..18: mol_type = other DNA
gctagctcggctttcctc

SEQ ID NO: 3 - DNA, 35 bp, synthetic construct
  source 1..35: mol_type = other DNA
ttggctgccgagggactgccagcggcggaacctgt

SEQ ID NO: 4 - DNA, 42 bp, synthetic construct
  source 1..42: mol_type = other DNA
tcgagtggtgtacacggaccacggatggtgatgtagcgactg

SEQ ID NO: 5 - DNA, 17 bp, synthetic construct
  source 1..17: mol_type = other DNA
ggcttccgcatccactc

SEQ ID NO: 6 - DNA, 17 bp, synthetic construct
  source 1..17: mol_type = other DNA
cggctggagctggagaa

SEQ ID NO: 7 - DNA, 16 bp, synthetic construct
  source 1..16: mol_type = other DNA
cagcggcggaacctgt

SEQ ID NO: 8 - DNA, 19 bp, synthetic construct
  source 1..19: mol_type = other DNA
ttggctgccgagggactgc

SEQ ID NO: 9 - DNA, 20 bp, synthetic construct
  source 1..20: mol_type = other DNA
ggatggtgatgtagcgactg

SEQ ID NO: 10 - DNA, 22 bp, synthetic construct
  source 1..22: mol_type = other DNA
tcgagtggtgtacacggaccac

SEQ ID NO: 11 - DNA, 18 bp, synthetic construct
  source 1..18: mol_type = other DNA
ggtgtacacggaccacca

SEQ ID NO: 12 - DNA, 20 bp, synthetic construct
  source 1..20: mol_type = other DNA
gctgctgcaacttcttcttg

SEQ ID NO: 13 - DNA, 40 bp, synthetic construct
  source 1..40: mol_type = other DNA
ctcggctttcctccggatggctggagctggagaaggagtt

SEQ ID NO: 14 - DNA, 37 bp, synthetic construct
  source 1..37: mol_type = other DNA
acgctggggctctctgagactctcctttgctctgcgg

SEQ ID NO: 15 - DNA, 22 bp, synthetic construct
  source 1..22: mol_type = other DNA
ggcaggttaaaatctggtttca

SEQ ID NO: 16 - DNA, 20 bp, synthetic construct
  source 1..20: mol_type = other DNA
ctggagctggagaaggagtt

SEQ ID NO: 17 - DNA, 20 bp, synthetic construct
  source 1..20: mol_type = other DNA
ctcggctttcctccggatgg

SEQ ID NO: 18 - DNA, 20 bp, synthetic construct
  source 1..20: mol_type = other DNA
ctctcctttgctctgcggtt

SEQ ID NO: 19 - DNA, 19 bp, synthetic construct
  source 1..19: mol_type = other DNA
acgctggggctctctgaga

SEQ ID NO: 20 - DNA, 18 bp, synthetic construct
  source 1..18: mol_type = other DNA
agtaccccatcgagcacg

SEQ ID NO: 21 - DNA, 20 bp, synthetic construct
  source 1..20: mol_type = other DNA
agcctggatagcaacgtaca

SEQ ID NO: 22 - DNA, 40 bp, synthetic construct
  source 1..40: mol_type = other DNA
gagccacacgcagctcattgtatcaccaactgggacgaca

SEQ ID NO: 23 - DNA, 38 bp, synthetic construct
  source 1..38: mol_type = other DNA
ctgaaccccaaggccaaccggctggggtgttgaaggtc

SEQ ID NO: 24 - DNA, 21 bp, synthetic construct
  source 1..21: mol_type = other DNA
tgtggtgccagattttctcca

SEQ ID NO: 25 - DNA, 23 bp, synthetic construct
  source 1..23: mol_type = other DNA
cgagaagatgacccagatcatgt

SEQ ID NO: 26 - DNA, 18 bp, synthetic construct
  source 1..18: mol_type = other DNA
tcaccaactgggacgaca

SEQ ID NO: 27 - DNA, 22 bp, synthetic construct
  source 1..22: mol_type = other DNA
gagccacacgcagctcattgta

SEQ ID NO: 28 - DNA, 18 bp, synthetic construct
  source 1..18: mol_type = other DNA
gctggggtgttgaaggtc

SEQ ID NO: 29 - DNA, 20 bp, synthetic construct
  source 1..20: mol_type = other DNA
ctgaaccccaaggccaaccg

SEQ ID NO: 30 - DNA, 17 bp, synthetic construct
  source 1..17: mol_type = other DNA
gctcggctacagcttca

SEQ ID NO: 31 - DNA, 18 bp, synthetic construct
  source 1..18: mol_type = other DNA
ggaagagtgcctcagggc

SEQ ID NO: 32 - DNA, 38 bp, synthetic construct
  source 1..38: mol_type = other DNA
aagtccagggcgacgtagcaccggccggagcgggaaat

SEQ ID NO: 33 - DNA, 41 bp, synthetic construct
  source 1..41: mol_type = other DNA
gagatggccacggctgcttccattgccaatggtgatgacct

SEQ ID NO: 34 - DNA, 21 bp, synthetic construct
  source 1..21: mol_type = other DNA
ttctccttaatgtcacgcacg

SEQ ID NO: 35 - DNA, 19 bp, synthetic construct
  source 1..19: mol_type = other DNA
ccctgagaagagctacgag

SEQ ID NO: 36 - DNA, 16 bp, synthetic construct
  source 1..16: mol_type = other DNA
cggccgagcgggaaat

SEQ ID NO: 37 - DNA, 21 bp, synthetic construct
  source 1..21: mol_type = other DNA
aagtccagggcgacgtagcac

SEQ ID NO: 38 - DNA, 19 bp, synthetic construct
  source 1..19: mol_type = other DNA
ttgccaatggtgatgacct

SEQ ID NO: 39 - DNA, 22 bp, synthetic construct
  source 1..22: mol_type = other DNA
gagatggccacggctgcttcca

SEQ ID NO: 40 - DNA, 19 bp, synthetic construct
  source 1..19: mol_type = other DNA
aaacctgtgcgagtggatg

SEQ ID NO: 41 - DNA, 18 bp, synthetic construct
  source 1..18: mol_type = other DNA
tcggagagcccaagtgtg

SEQ ID NO: 42 - DNA, 40 bp, synthetic construct
  source 1..40: mol_type = other DNA
ggtctgtgtacaccacccggtagcagtccctaggaagcca

SEQ ID NO: 43 - DNA, 40 bp, synthetic construct
  source 1..40: mol_type = other DNA
ggctggagctggagaaggagtgccagctcacttttcctcc

SEQ ID NO: 44 - DNA, 22 bp, synthetic construct
  source 1..22: mol_type = other DNA
tgtcttttgtcctggttttcac

SEQ ID NO: 45 - DNA, 25 bp, synthetic construct
  source 1..25: mol_type = other DNA
cactttagtcgatacatcaccatca

SEQ ID NO: 46 - DNA, 18 bp, synthetic construct
  source 1..18: mol_type = other DNA
gcagtccctaggaagcca

SEQ ID NO: 47 - DNA, 22 bp, synthetic construct
  source 1..22: mol_type = other DNA
ggtctgtgtacaccacccggta

SEQ ID NO: 48 - DNA, 19 bp, synthetic construct
  source 1..19: mol_type = other DNA
gccagctcacttttcctcc

SEQ ID NO: 49 - DNA, 21 bp, synthetic construct
  source 1..21: mol_type = other DNA
ggctggagctggagaaggagt

SEQ ID NO: 50 - DNA, 21 bp, synthetic construct
  source 1..21: mol_type = other DNA
agcggcgaaacctgtgcgagt

SEQ ID NO: 51 - DNA, 18 bp, synthetic construct
  source 1..18: mol_type = other DNA
accaccatgtacccaggc

SEQ ID NO: 52 - DNA, 18 bp, synthetic construct
  source 1..18: mol_type = other DNA
ggccggactcatcgtact

SEQ ID NO: 53 - DNA, 42 bp, synthetic construct
  source 1..42: mol_type = other DNA
gcgctcaggaggagcaatgatcattgctgacaggatgcagaa

SEQ ID NO: 54 - DNA, 42 bp, synthetic construct
  source 1..42: mol_type = other DNA
tactctgtgtggatcggtggcttgcttgctgatccacatctg

SEQ ID NO: 55 - DNA, 18 bp, synthetic construct
  source 1..18: mol_type = other DNA
tggtgctaggagccagag

SEQ ID NO: 56 - DNA, 19 bp, synthetic construct
  source 1..19: mol_type = other DNA
cctcactgtccaccttcca

SEQ ID NO: 57 - DNA, 20 bp, synthetic construct
  source 1..20: mol_type = other DNA
attgctgacaggatgcagaa

SEQ ID NO: 58 - DNA, 22 bp, synthetic construct
  source 1..22: mol_type = other DNA
gcgctcaggaggagcaatcatc

SEQ ID NO: 59 - DNA, 20 bp, synthetic construct
  source 1..20: mol_type = other DNA
tgcttgctgatccacatctg

SEQ ID NO: 60 - DNA, 22 bp, synthetic construct
  source 1..22: mol_type = other DNA
tactctgtgtggatcggtggct

SEQ ID NO: 61 - DNA, 20 bp, synthetic construct
  source 1..20: mol_type = other DNA
cctgtggcatccatgaaact

SEQ ID NO: 62 - DNA, 18 bp, synthetic construct
  source 1..18: mol_type = other DNA
ggacagtgaggccaggat

SEQ ID NO: 63 - DNA, 42 bp, synthetic construct
  source 1..42: mol_type = other DNA
ctgggtacatggtggtaccaccaagtgtgacgttgacatccg

SEQ ID NO: 64 - DNA, 41 bp, synthetic construct
  source 1..41: mol_type = other DNA
tactgctctggctcctagcaccagagtacttgcgctcagga

SEQ ID NO: 65 - DNA, 22 bp, synthetic construct
  source 1..22: mol_type = other DNA
tgtgttggcatagaggtcttta

SEQ ID NO: 66 - DNA, 22 bp, synthetic construct
  source 1..22: mol_type = other DNA
tgaagatcaagatcattgctcc

SEQ ID NO: 67 - DNA, 20 bp, synthetic construct
  source 1..20: mol_type = other DNA
aagtgtgacgttgacatccg

SEQ ID NO: 68 - DNA, 22 bp, synthetic construct
  source 1..22: mol_type = other DNA
ctgggtacatggtggtaccacc

SEQ ID NO: 69 - DNA, 19 bp, synthetic construct
  source 1..19: mol_type = other DNA
agagtacttgcgctcagga

SEQ ID NO: 70 - DNA, 22 bp, synthetic construct
  source 1..22: mol_type = other DNA
tactgctctggctcctagcacc

(19) United States
(12) Patent Application Publication
(10) Pub. No.: US 2025/0179581 A1
(43) Pub. Date: Jun. 5, 2025

(54) MEASUREMENT OF NUCLEIC ACIDS IN A BIOLOGICAL SPECIMEN USING RT-LAMP

(71) Applicant: The Regents of the University of California, Oakland, CA (US)

(72) Inventor: Debashis Sahoo, San Diego, CA (US)

(21) Appl. No.: 18/840,003

(22) PCT Filed: Feb. 21, 2023
(86) PCT No.: PCT/US2023/013507

§ 371 Date: Aug. 20, 2024

Related U.S. Application Data
(60) Provisional application No. 63/312,268, filed on Feb. 21, 2022.

(51) Int. Cl. C12Q 1/6886

(52) U.S. Cl. CPC C12Q 1/6886; C12Q 2600/158

(57) ABSTRACT

Methods and devices for utilizing reverse transcription loop-mediated isothermal amplification (RT-LAMP) to detect target DNA and RNA sequences for diagnostic and experimental assays, such as those for diagnosing and quantifying diseases, such as colorectal cancer and gastrointestinal disease, or pathogens, such as SARS-COV-2.

### CROSS-REFERENCE TO RELATED APPLICATIONS

This application claims priority to U.S. Provisional Application No. 63/312,268, filed on Feb. 21, 2022, the entire contents of which are incorporated by reference.

### GOVERNMENT SPONSORSHIP

This invention was made with government support under Grant Nos. R01-GM138385, R01-AI155696, and UG3-TR003355 awarded by the National Institutes of Health. The government has retained certain rights in the invention.

### SEQUENCE LISTING

The instant application contains a Sequence Listing which has been submitted electronically in ASCII format and is hereby incorporated by reference in its entirety. Said ASCII copy, created on 20_, is named SL.txt and is bytes in size.

### FIELD OF THE INVENTION

The presently disclosed inventive concepts relate generally to biological and environmental specimen sampling of nucleotides of interest, including for diagnostic purposes.

### BACKGROUND

Loop mediated isothermal amplification (LAMP) protocols¹ have become extremely popular method of SARS-COV-2 diagnostic assay. Diagnostics based on LAMP amplification are a compelling alternative to polymerase chain reaction (PCR) because LAMP can be performed without the need for commercial thermocyclers, resulting in decreased time to result^{1,2}. The simplicity of isothermal amplification and the absence of separate steps for mRNA extraction allow for translation to a simple point-of-care device based on disposable cartridges³⁻⁵. In addition, LAMP has advantages over PCR for targeting sequences, due to its robustness against inhibitors^{6,7} as well as its high specificity, using four to six primers that identify six to eight regions on the template for amplification¹. LAMP assay kits are commercially available but almost all of them use LAMP for detection as opposed to reliable quantification directly from sample⁸⁻¹⁴. Reverse transcription-polymerase chain reaction (RT-PCR) is a commonly used laboratory techniques for reliable quantification of nucleic acid and considered gold standard for this application.

Reverse transcription-polymerase chain reaction (RT-PCR) is a commonly used laboratory techniques for gene expression analysis. It uses an enzyme called reverse transcriptase to change a specific piece of RNA into a matching piece of DNA. This piece of DNA is then amplified (made in large numbers) by another enzyme called DNA polymerase. The amplification kinetics of DNA copies help quantify a specific mRNA molecule that is being made by a gene. Reverse transcription-loop mediated isothermal amplification (RT-LAMP) uses an alternative method of DNA amplification based on DNA polymerase with strand displacement activity, which eliminates the DNA denaturation stage². The technique is highly specific and increases the amount of amplified DNA even up to a billion copies over less than an hour, compared to a million copies yielded by the PCR. Isothermal amplification can be performed without advanced laboratory equipment, such as in a dry block heater or a water bath. Early reports suggest that the diagnostic accuracy of RT-PCR is much better than RT-LAMP¹³.

The detection of changes in fluorescence intensity during the reaction enables the user to follow the PCR reaction in real time. RT-PCR comprises several steps: (1) RNA is isolated from target tissue/cells; (2) mRNA is reverse-transcribed to cDNA; (3) modified gene-specific PCR primers are used to amplify a segment of the cDNA of interest, following the reaction in real time; and (4) the initial concentration of the selected transcript in a specific tissue or cell type is calculated from the exponential phase of the reaction. Relative quantification or absolute quantification compared to standards that are run in parallel can be performed. RT-PCR technique is more expensive (˜$25/sample for reagents) and takes several days. On the other hand, reverse transcription loop-mediated isothermal amplification (RT-LAMP) has been for point-of-need diagnostic testing for several pathogens, including SARS-CoV-2^{5,6,13,14} RT-LAMP assays are an alternative method for rapidly detecting the presence of specific nucleic acids in samples, with colorimetric or fluorescent visualization of results. RT-LAMP assays are inexpensive (˜$7/sample), high-throughput (can be run in a 96-well format), do not necessarily require nucleic acid purification, and give rapid results (˜60-90 minutes from setup to results).

### SUMMARY OF THE INVENTION

Disclosed herein are methods and devices for utilizing reverse transcription loop-mediated isothermal amplification (RT-LAMP) to detect target DNA and RNA sequences to provide diagnostic and experimental assays, such as those for diagnosing and quantifying human diseases, such as colorectal cancer, or pathogens, such as SARS-COV-2. The methods and devices disclosed herein utilize a unique, customizable set of primers to improve the accuracy of RT-LAMP for target nucleic acid quantification.

In embodiments, the disclosure provides a method for identifying a plurality of primers for identifying a target nucleic acid in a biological sample using reverse transcription loop-mediated isothermal amplification (RT-LAMP), where the method includes providing a nucleotide sequence for the target nucleic acid to a computer program, where the computer program is configured to identify the plurality of primers based on the nucleotide sequence for the target nucleic acid. In some embodiments, the method provides 4-6 primers optimized for quantifying target DNA or RNA sequences in a biological sample, such as blood, saliva or tissues taken from a human patient.

In embodiments, the disclosure provides a method of detecting a nucleic acid in a biological specimen, where the method includes collecting a biological sample, preparing a test sample, the test sample comprising the biological sample and a plurality of primers, providing the test sample to a device configured to perform reverse transcription loop-mediated isothermal amplification (RT-LAMP), amplifying the nucleic acid sequence using RT-LAMP, and determining, from the amplification, whether and in what amount the nucleic acid sequence is present in the biological sample. In some embodiments, the method is effective to diagnose and quantify diseases, such as colorectal cancer and gastrointestinal disease, or pathogens, such as SARS-COV-2.

In embodiments, the disclosure provides a desktop reverse transcription loop-mediated isothermal amplification (RT-LAMP) device, where the device includes a housing defining a slot and a sample holder therein, a display affixed to an exterior surface of the housing, and a sample processing package disposed within the housing, where the device is configured to detect the presence of a nucleic acid in a biological sample. In some embodiments, the device may be used to diagnose and quantify diseases, such as colorectal cancer and gastrointestinal disease, or pathogens, such as SARS-COV-2.

### BRIEF DESCRIPTION OF THE DRAWINGS

FIGS. 1A-1B. Workflow for faster measurement of mRNA expression. FIG. 1A. Schematic displays of the proposed direct sample testing using RT-LAMP for quick expression measurement in a colon tissue. FIG. 1B. Proposed device and workflow to demonstrate mRNA expression measurement at home.

FIGS. 2A-2C. mRNA expression measurement. FIG. 2A. Proposed experiment to demonstrate CDX2 differential expression patterns in blood, PBMC, lung, kidney, spleen, and colon tissue. FIG. 2B. Proposed experiment to test expression patterns starting from cDNA, mRNA and tissue quick extract samples. FIG. 2C. Proposed experiment to compare RT-PCR and RT-LAMP techniques for quantitative expression measurement using serial dilution samples.

FIGS. 3A-3D. Primers design. Visualization of the RT-LAMP primer design (F3, F2, LF, F1c, B1c, B2, B3), steps with gene model based on latest genome, and RefSeq transcript with exon annotations. Two different primers were designed for each gene. Location of the primers were specified in the target sequence. FIG. 3A. Primers for CDX2. FIG. 3B. Primers for ACTB. FIG. 3C. Primers for Mouse Cdx2 gene. FIG. 3D. Primers for Actb gene.

FIGS. 4A-4C. Rapid CDX2 expression measurement using RT-LAMP and comparison with RT-PCR. FIG. 4A. Five parameter logistic (5PL) model for comparative analysis of RT-PCR and RT-LAMP data. 5PL model parameters: baseline (a), slope factor (b), inflection point (c), plateau (d) and asymmetry factor (e) are displayed for a sample RT-PCR (left) and RT-LAMP (right) data. X-axis displays time in seconds and y-axis displays the raw fluorescence intensity. 5PL model is computed using least squares regression and the model predicted data is shown as a blue line on top of the original raw data as grey dots. Inflection point is shown using a vertical red line. FIG. 4B. RT-LAMP experiments using hACTB, ACTB2, CDX2 and hCDX2 RT-LAMP primer sets on three different human tissues (lung, colon and blood) cDNA samples. FIG. 4C. RT-LAMP experiment is performed on cDNA, mRNA and Tissue quick extract samples. Welch's two sample two-tailed unpaired t-test is performed to compute the p values.

FIG. 5. Rapid mRNA expression measurement using RT-LAMP and comparison with RT-PCR. Human normal colon cDNA samples were used in RT-PCR and RT-LAMP experiments. hACTB and hCDX2 LAMP primers set were used for the RT-LAMP experiment. F3 and B3 primers (hACTB and hCDX2) were used to perform RT-PCR experiment. RT-PCR data is directly compared to RT-LAMP data on the same plot with same time scale and normalized fluorescence intensity ranging from 0 to 1. RAW fluorescence intensities, 5PL b and e values are compared between RT-PCR and RT-LAMP using violin plots.

FIGS. 6A-6B. Direct tissue sample testing. FIG. 6A. RT-LAMP experiments on direct tissue sample from five different mouse tissues (colon, lung, kidney, spleen, blood) using all four primer sets: Actb (top-left), Actb2 (top-right), Cdx2 (bottom-left), and mCdx2 (bottom-right). FIG. 6B. RT-LAMP experiment performed on colon samples prepared using 10 different protocols and two primer sets (Actb, mCdx2). CS1 is collected using a cotton swab on the opposite size of the colon epithelium (-ve control for Cdx2 expression) processed using Lucigen QuickExtract solution. CS2 is collected using a cotton swab directly on the colon epithelium. C is a big chunk of whole colon tissue dropped in Lucigen QuickExtract solution. CS1n and CS2n is the same CS1 and CS2 samples stored in −20 C for 3 days respectively and re-used for the RT-LAMP experiment. QE is same as sample C stored in −20 C for 3 days and re-used for the RT-LAMP experiment. A tiny chunk of colon tissue is transferred to a new QuickExtract solution from sample C stored in −20 C for 3 days for QE2 sample. A whole chunk of colon tissue was stored in RNALater in −20 C for 3 days and re-used to make RLS1, RLQE1 and RLQE2 samples. RLS1 is collected using a cotton swab on the whole colon chunk from the RNALater solution. A small chunk of colon tissue from the RNALater solution is dropped into fresh QuickExtract solution to make RLQE1 sample and processed quickly after 1 minute. RLQE2 sample is same as RLQE1 sample processed after 20 minutes.

FIGS. 7A-7B. Correlation between RT-PCR and RT-LAMP. FIG. 7A. Serial dilution was used to prepare human colon cDNA samples in various concentration. hACTB and hCDX2 LAMP primer sets were used to perform RT-LAMP experiment from the diluted samples. F3 and B3 primers (hACTB and hCDX2) were used to perform RT-PCR experiment on the same diluted samples. 5PL model was used to identify the inflection point for both RT-LAMP and RT-PCR data and visualized using a scatter plot. This experiment was repeated in two different human cDNA samples (left and right). Correlation tests between RT-PCR and RT-LAMP data for both primers were calculated and displayed as scatter plots using python seaborn Implots with the p-values. The confidence interval around the regression line is indicated with shades. FIG. 7B. 3D-model (4″×3″×5″) of the 3D-printed device and assembly details for use at home and outdoor setting. It uses Arduino Pro Mini microcontroller and ESP32-CAM camera module to detect fluorescence signal. Results are displayed in a 0.96″ 128×64 OLED LCD Display. Data from the device is collected over WIFI using ESP32 module to a cell phone or a computer.

FIG. 8. Relationship between RT-LAMP quantification and F3/B3 RT-PCR. Eleven different RT-LAMP primers (Supplementary Data 1, 3) are used on six different serial dilutions of colon mRNA samples. Correlation coefficient between the inflection points and dilutions is plotted on the x-axis. Y-axis represent the RT-PCR inflection point (Ct) of F3/B3 parts of the RT-LAMP primers. Each point in the plot represents a RT-LAMP primers.

### DETAILED DESCRIPTION

The presently disclosed inventive concepts are directed to a method and apparatus for sampling, extracting, and capturing analyte macromolecules of interest, such as nucleic acids, from a biological specimen. The disclosure includes the processes and devices for sampling a volume of material, including the nucleic acids of interest, from the biological specimen for further detection and quantitative analysis.

Reverse transcription loop-mediated isothermal amplification (RT-LAMP) is a versatile technique for detection of target DNA and RNA, which enables development of diagnostic assays at the point of care setting. The global SARS-COV-2 pandemic has enabled rapid development of SARS-COV-2 detection kits using RT-LAMP protocols. The sensitivity of RT-LAMP in early reports has been below that of the standard RT-PCR tests, and there are numerous attempts to improve performance. The invention disclosed herein provides the use of a fluorescence-based RT-LAMP protocol to measure expression patterns of markers such as CDX2 or ACTB for diagnosing and quantifying diseases, such as colorectal cancer and gastrointestinal disease, or pathogens, such as SARS-COV-2.

The data demonstrates that this simple method matches extremely well to the standards of sophisticated RT-PCR techniques (r=0.99, p<0.001), and the assay works on diverse sample types such as cDNA, mRNA, and direct tissue sample testing. In embodiments, it is estimated that the time from sample collection to CDX2 expression data is 25 minutes, which is significantly less than the current standards (RT-PCR will be more than 3 hours). This enables quick diagnostic assay at the point of care setting with less costly equipment.

The invention disclosed herein provides that RT-LAMP can be used to measure gene expression values robustly and reliably. RT-LAMP technologies typically require complex primer design steps, and self-amplification of these primers often leads to false positive results. However, the present disclose provides a new protocol for improved primer design, which ultimately reduce the overall time for tissue sampling and testing.

The invention disclosed herein leverages the advantages of the RT-LAMP to develop molecular diagnostic assays based on target nucleic acid expression in a biological sample, such as CDX2 expression. Cotton swab-based sample collection and direct sample testing procedures were developed which was demonstrated to be superior to whole tissue chunks. The time from sample collection to CDX2 expression data can be about 25 minutes. Future optimization on the primer set can reduce this time further. A 10-25 minute diagnostic assay at the point of care setting will improve health care broadly across many diseases and pathogens. The invention also provides a device for portable at home and remote molecular testing.

The invention provides methods for signal detection for gene expression quantification for target gene marker expression, such as for CDX2 and ACTB, which are useful for gastrointestinal disease and colorectal cancer diagnostics. However, the methods can be easily generalizable to many other genes, including pathogens, such a SARS-COV-2.

In embodiments, methods for identifying primers useful for identifying a target nucleic acid in a biological sample using reverse transcription loop-mediated isothermal amplification (RT-LAMP). The method includes providing a nucleotide sequence for the target nucleic acid to a computer program, where the computer program identifies the plurality of primers based on the nucleotide sequence of the target nucleic acid. In some embodiments, the primers are identified based on the concentration of nucleotide pairs in the nucleic acid sequence, the location of nucleotide pairs in the nucleic acid sequence, and/or the distance between DNA regions in the nucleic acid sequence, as described in the examples herein.

In some embodiments, a plurality of primers are used, preferably 4-6 primers. These primers include internal primers, external primers, and loop primers, which may be forward and/or backward internal primers, forward and/or backward primers, and loop forward and/or loop backward primers. In some embodiments, the forward internal primers are F1c::F2 and the backward internal primers are B2::B2c. In other embodiments, the forward primer is F3 and the backward primer is B3. In further embodiments, the loop forward primer is LF and the loop backward primer is LB. In a preferred embodiment, each primer is a single strand structure at a temperature from about 60° C. to about 65° C.

In embodiments, a method of detecting a nucleic acid in a biological specimen is provided. The method includes the steps of collecting a biological sample, preparing a test sample having the biological sample and a plurality of primers, providing the test sample to a device configured to perform reverse transcription loop-mediated isothermal amplification (RT-LAMP), amplifying the nucleic acid sequence using RT-LAMP, and determining, from the amplification, in what quantity the nucleic acid sequence is present in the biological sample.

In some embodiments, the biological specimen includes, but is not limited to, a human fluid or tissue, bacteria, virus, archaea, fungi, and/or biofilm. In an embodiment, the biological specimen is colon tissue and the primers are selected from target nucleic acid sequences in CDX2 and ACTB genes. The method may therefore be effective to diagnose a patient from which the biological sample is collected with gastrointestinal diseases and colorectal cancer. In some embodiments, the biological sample includes, but is not limited to, human tissue, saliva, blood, hair, urine, and/or bodily secretions.

In some embodiments, the plurality of primers include 4-6 primers which may be any combination of forward internal primers, backward internal primers, forward primers, backward primers, loop forward primers, and loop backward primers. The plurality of primers are selected based on a target nucleotide sequence of the nucleic acid.

In embodiments, a desktop reverse transcription loop-mediated isothermal amplification (RT-LAMP) device is provided, the device including a housing defining a slot and a sample holder therein, a display affixed to an exterior surface of the housing, and a sample processing package disposed within the housing. In some embodiments, the device is configured to detect the presence of a nucleic acid sequence in a biological sample.

In some embodiments, the sample processing package of the portable desktop RT-LAMP device includes a filter, camera, heater, and processor. In some embodiments, the processor is configured to receive a user input, execute a command for performing RT-LAMP in accordance with the user input, process an output from the camera, and display the output on the display. In some embodiments, the filter and the camera are positioned above the sample holder within the housing. In some embodiments, the desktop RT-LAMP device has dimensions less than 5 inches tall by 5 inches wide by 5 inches deep, such that the housing is less than 125 in³. According to one embodiment, the device is 5 inches tall, 4 inches wide, and 3 inches deep.

In some embodiments, a method of using a desktop RT-LAMP device is provided, where the method includes the steps of preparing a test sample comprising a biological sample and a plurality of primers, providing the test sample to the sample holder within the housing, supplying an input to the device, and receiving an output via the display, where the output may indicate whether the nucleic acid is present in the biological sample. In an embodiment, the desktop RT-LAMP device is configured to detect and quantify CDX2 which is useful for gastrointestinal diseases and colorectal cancer diagnosis.

Before further describing various embodiments of the presently disclosed inventive concepts in more detail by way of exemplary description, examples, and results, it is to be understood that the presently disclosed inventive concepts are not limited in application to the details of methods and compositions as set forth in the following description. The presently disclosed inventive concepts are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the presently disclosed inventive concepts may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure.

All patents, published patent applications, and non-patent publications mentioned in the specification are indicative of the level of skill of those skilled in the art to which the presently disclosed inventive concepts pertain. All patents, published patent applications, and non-patent publications referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the presently disclosed inventive concepts shall have the meanings that are commonly understood by those having ordinary skill in the art. Further, unless otherwise required by context, singular terms shall include pluralities and plural terms shall include the singular.

As utilized in accordance with the methods, compositions, and components of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and exemplary claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

In certain embodiments, the nucleic acids of interest are DNA or RNA. “Nucleic acid” or “nucleic acid molecule” refers to a multimeric compound comprising two or more covalently bonded nucleosides or nucleoside analogs having nitrogenous heterocyclic bases, or base analogs, where the nucleosides are linked together by phosphodiester bonds or other linkages to form a polynucleotide. Nucleic acids include RNA, DNA, or chimeric DNA-RNA polymers or oligonucleotides, and analogs thereof. A nucleic acid backbone can be made up of a variety of linkages, including one or more of sugar-phosphodiester linkages, peptide-nucleic acid bonds, phosphorothioate linkages, methylphosphonate linkages, or combinations thereof. Sugar moieties of the nucleic acid can be ribose, deoxyribose, or similar compounds having known substitutions (e.g. 2′-methoxy substitutions and 2′-halide substitutions). Nitrogenous bases can be conventional bases (A, G, C, T, U) or analogs thereof (e.g., inosine, 5-methylisocytosine, isoguanine). A nucleic acid can comprise only conventional sugars, bases, and linkages as found in RNA and DNA, or can include conventional components and substitutions (e.g., conventional bases linked by a 2′-methoxy backbone, or a nucleic acid including a mixture of conventional bases and one or more base analogs). Nucleic acids can include “locked nucleic acids” (LNA), in which one or more nucleotide monomers have a bicyclic furanose unit locked in an RNA mimicking sugar conformation, which enhances hybridization affinity toward complementary sequences in single-stranded RNA (ssRNA), single-stranded DNA (ssDNA), or double-stranded DNA (dsDNA). Nucleic acids can include modified bases to alter the function or behavior of the nucleic acid (e.g., addition of a 3′-terminal dideoxynucleotide to block additional nucleotides from being added to the nucleic acid). Synthetic methods for making nucleic acids in vitro are well known in the art although nucleic acids can be purified from natural sources using routine techniques. Nucleic acids can be single-stranded or double-stranded.

The biological specimen of the present disclosure can be selected from the any material that contains macromolecules, such as nucleic acids, including the group consisting of bacteria, viruses, archaea, fungi, algae, mammalian cells, plant cells, biofilms, plant tissue, animal tissue, human tissue, blood, saliva, hair, urine, tears, semen, other bodily secretions, biology-derived deposits, and combinations thereof. In certain embodiments, the biological specimen can comprise less than 10,000 cells, 1,000 cells, 100 cells, 10 cells, or a single cell. In certain embodiments, the biological specimens can be presented on solid or porous surfaces (such as a filter), and can be either wet or dry.

“Primer” refers to an enzymatically extendable oligonucleotide, generally with a defined sequence that is designed to hybridize in an antiparallel manner with a complementary, primer-specific portion of a target nucleic acid. A primer can initiate the polymerization of nucleotides in a template-dependent manner to yield a nucleic acid that is complementary to the target nucleic acid when placed under suitable nucleic acid synthesis conditions (e.g. a primer annealed to a target can be extended in the presence of nucleotides and a DNA/RNA polymerase at a suitable temperature and pH). Suitable reaction conditions and reagents are known to those of ordinary skill in the art. A primer is typically single stranded for maximum efficiency in amplification, but may alternatively be double stranded. If double stranded, the primer is generally first treated to separate its strands before being used to prepare extension products. The primer generally is sufficiently long to prime the synthesis of extension products in the presence of the inducing agent (e.g. polymerase). Specific length and sequence will be dependent on the complexity of the required DNA or RNA targets, as well as on the conditions of primer use such as temperature and ionic strength. Preferably, the primer is about 5-100 nucleotides. Thus, a primer can be, e.g., 5, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95 or 100 nucleotides in length. A primer does not need to have 100% complementarity with its template for primer elongation to occur; primers with less than 100% complementarity can be sufficient for hybridization and polymerase elongation to occur. A primer can be labeled if desired. The label used on a primer can be any suitable label, and can be detected by, for example, spectroscopic, photochemical, biochemical, immunochemical, chemical, or other detection means. A labeled primer therefore refers to an oligomer that hybridizes specifically to a target sequence in a nucleic acid, or in an amplified nucleic acid, under conditions that promote hybridization to allow selective detection of the target sequence.

A primer nucleic acid can be labeled, if desired, by incorporating a label detectable by, e.g., spectroscopic, photochemical, biochemical, immunochemical, chemical, or other techniques. To illustrate, useful labels include radioisotopes, fluorescent dyes, electron-dense reagents, enzymes (as commonly used in ELISAs), biotin, or haptens and proteins for which antisera or monoclonal antibodies are available. Many of these and other labels are described further herein and/or are otherwise known in the art. One of skill in the art will recognize that, in certain embodiments, primer nucleic acids can also be used as probe nucleic acids.

“Region” refers to a portion of a nucleic acid wherein said portion is smaller than the entire nucleic acid.

“Region of interest” refers to a specific sequence of a target nucleic acid that includes all codon positions having at least one single nucleotide substitution mutation associated with a genotype and/or subtype that are to be amplified and detected, and all marker positions that are to be amplified and detected, if any.

“RNA-dependent DNA polymerase” or “reverse transcriptase” (“RT”) refers to an enzyme that synthesizes a complementary DNA copy from an RNA template. All known reverse transcriptases also have the ability to make a complementary DNA copy from a DNA template; thus, they are both RNA- and DNA-dependent DNA polymerases. RTs may also have an RNAse H activity. A primer is required to initiate synthesis with both RNA and DNA templates.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

It will be understood from the foregoing description that various modifications and changes may be made in the various embodiments of the present disclosure without departing from their true spirit. The description provided herein is intended for purposes of illustration only and is not intended to be construed in a limiting sense. Thus, while the presently disclosed inventive concepts have been described herein in connection with certain embodiments so that aspects thereof may be more fully understood and appreciated, it is not intended that the presently disclosed inventive concepts be limited to these particular embodiments. On the contrary, it is intended that all alternatives, modifications and equivalents are included within the scope of the presently disclosed inventive concepts as defined herein. Thus the examples described above, which include particular embodiments, will serve to illustrate the practice of the presently disclosed inventive concepts, it being understood that the particulars shown are by way of example and for purposes of illustrative discussion of particular embodiments of the presently disclosed inventive concepts only and are presented in the cause of providing what is believed to be a useful and readily understood description of procedures as well as of the principles and conceptual aspects of the inventive concepts. Changes may be made in the construction and formulation of the various components and compositions described herein, the methods described herein or in the steps or the sequence of steps of the methods described herein without departing from the spirit and scope of the presently disclosed inventive concepts.

### EXAMPLES

### Example 1. Materials and Methods

### Tissue Samples

Human normal colon cDNA and mRNA samples were purchased from AMSBIO LLC (Colon cDNA #C1234090; Colon mRNA #ATR1234090-50) and Takara Bio (cDNA #639331). Human Lung cDNA samples were purchased from Takara Bio (Lung cDNA #639308). Human blood samples were collected as part of University of California San Diego IRB Project #171861 and patients provided written informed consent.

Fresh tissues (Colon, Lung, Kidney, Spleen) of C57BI/6J mice were collected in PBS for immediate processing or in RNALater and stored in −20° C. Cardiac puncture from euthanized mouse was used to collect blood sample and immediately transferred to Lucigen QuickExtract solution or CPT tubes for PBMC isolation.

### RT-LAMP

RT-LAMP reactions (25 uL total volume) contained 1× WarmStart® Fluorescent LAMP/RT-LAMP Kit with UDG (New England Biolabs, Ipswich, MA, USA; #E1708L), a primer set composed of 1.6 uM FIP/BIP internal primers, 0.4 uM LF/LB loop primers and 0.2 uM F3/B3 external primers, and 2 μL of target samples. RT-LAMP reactions were carried out at 65° C. for 45 min.

### RT-PCR

A 20 μl reaction comprising 1 μl cDNA, a mixture of equal amounts of 0.5 uM forward and reverse primers and RT-PCR master mix as recommended by the manufacturer was used. Reaction conditions were initial denaturation at 95° C. for 10 min and 40 cycles consisting of 15 s at 95° C., 1 min at 60° C. and final extension 5 s at 65° C. Melt curve was produced from 65° C. to 95° C. with increment of 0.5° C. Runs were performed using a BioRad CFX96 RT-PCR system. PCR products were analyzed by electrophoresis on a 1.5% agarose gel, 0.5× TE; run at 100 V for 25 minutes, stained with GelRed and visualized using a UV transilluminator.

### Data Collection and Processing

A Python script was developed to analyze the SYBR green Quantification Amplification Results from BioRad CFX96. The raw fluorescence intensity was plotted as a function of time to generate the RT-LAMP amplification curve for each chamber. The amplification curve was analyzed using 5-parameters logistic model (y=d+ (a−d)/((1+(x/c)\hat{ }b)\hat{ }e) where a=baseline, b=slope factor, c=inflection point, d=plateau, e=asymmetry factor. Correlation tests between RT-PCR and RT-LAMP data was performed using Python Seaborn Package Implots function. Fluorescence intensity was normalized using (v-min)/max formula. RT-PCR and RT-LAMP curves were plotted using Python Pandas Package line plot function. Welch's two sample unpaired t-test is performed to compute the p values. Violin and swarm plots were made using Python Seaborn Package.

### Example 2. RT-LAMP Assay Quality Compared with RT-PCR

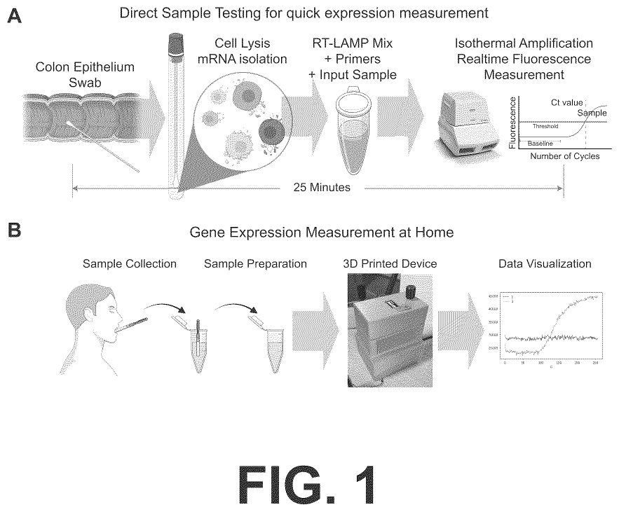

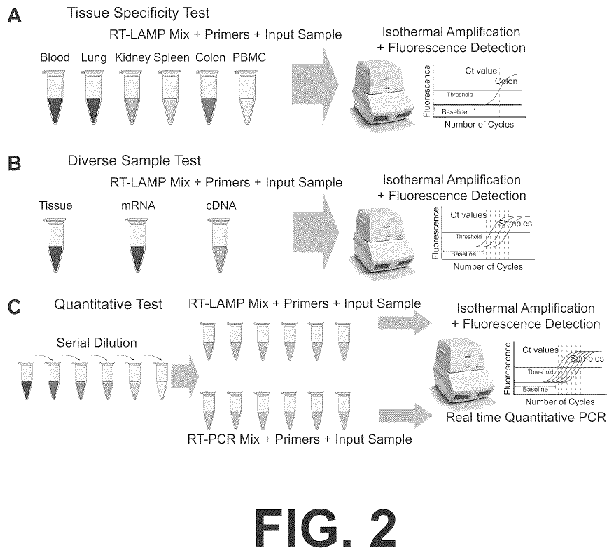

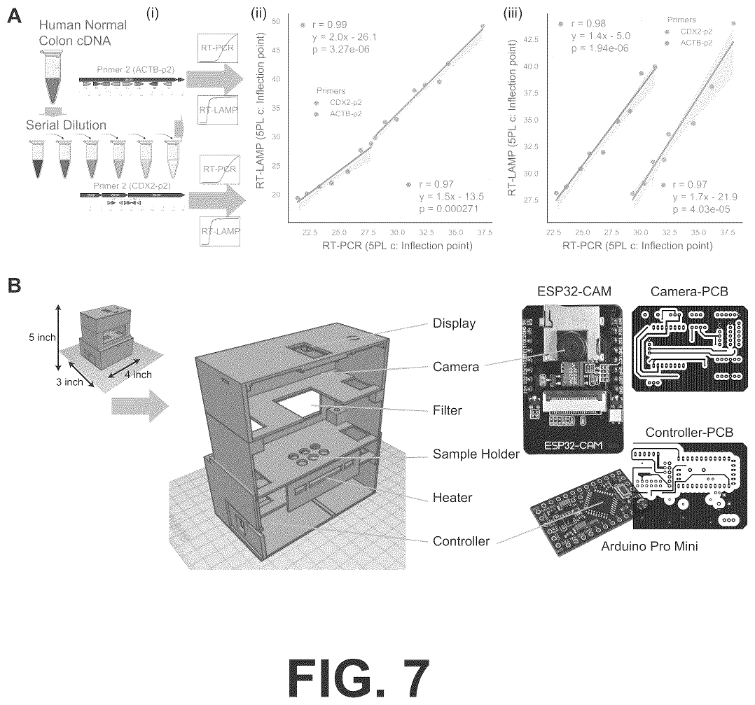

An RT-LAMP based approach was compared directly with RT-PCR for measuring CDX2 and ACTB gene expression values (FIGS. 1A-1B, FIGS. 2A-2C). A schematic experimental design to test RT-PCR and RT-LAMP data is shown in FIGS. 2A-2C. The time from sample collection to the fluorescent signal crossing the detection threshold was measured for both genes in both RT-PCR and RT-LAMP settings (FIG. 1A, FIG. 2A). Multiple tissue samples were tested in both human and mouse for tissue specific gene expression patterns (FIG. 2A). CDX2 is known to be colon tissue specific which is matched against the data from RT-PCR and RT-LAMP. Diverse sample types, such as cDNA, mRNA and Tissue QuickExtract, were tested in both settings (FIG. 2B). To check quantitative features for both RT-PCR and RT-LAMP data, a serial dilution experiment was performed. The Ct values were compared by using a correlation test (FIG. 2C). The goal of this experiment was to evaluate whether RT-LAMP data can be used reliably to measure gene expression. Whether the RT-LAMP assay can be performed at home was also tested by building a 3D-printed device (FIG. 7B).

### Example 3. Primer Design Steps for CDX2 and ACTB

Another innovative aspect of RT-LAMP is its high specificity due to the use of several primers (from four to six), which can distinguish up to eight specific locations on the DNA template, compared to only two in typical PCR. A deciding element responsible for the correct progression of the RT-LAMP reaction is the primer design stage. Several pairs of primers must be optimized in terms of a range of factors, including concentration, location of nucleotide pairs, and distance between DNA regions. The primers must have a single-strand structure at 60-65° C. and must not create a stable double-strand structure. Using a bigger number of primers to amplify, the same sequence can increase the interactions between them.

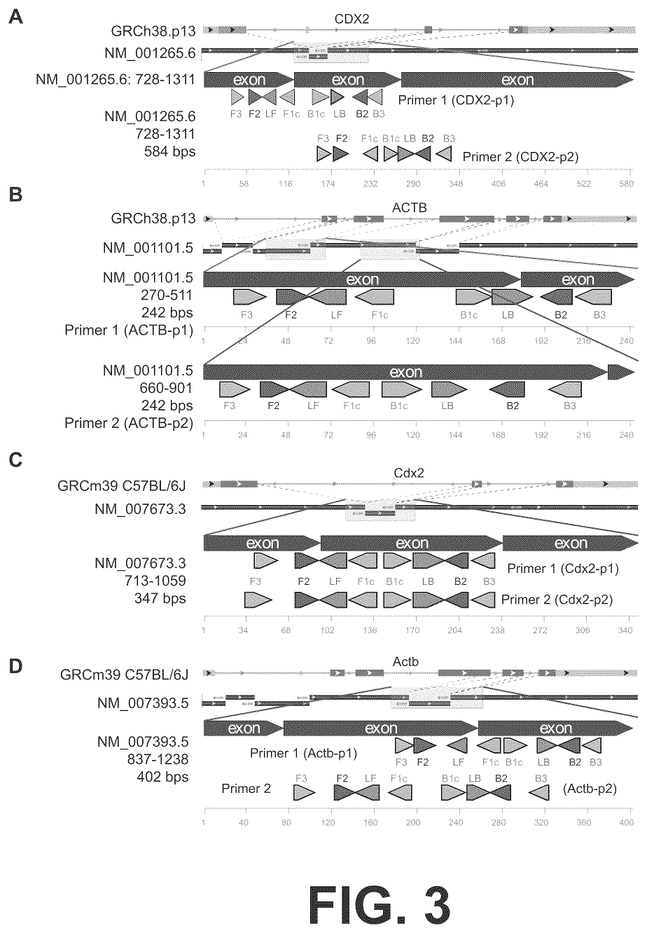

The design of RT-LAMP primers can be carried out using online software such as PrimerExplorer, Premier Biosoft, or NEB LAMP Primer Design Tool. The pairs of primers used in RT-LAMP are as follows: the internal primers, forward internal primer (FIP=F1c::F2) and backward internal primer (BIP=B2::B1c); the external primers, forward primer (F3) and backward primer (B3); and the optional loop primers, loop primer forward (LF) and loop primer backward (LB). To design a primer set for CDX2, sequence data from RefSeq NM 001265.6 was used (FIG. 3A). A target region that includes two exon boundaries (NM_001265.6:728-1311) was used to search for the RT-LAMP primers.

Two different primer sets (Primer 1: CDX2-p1, Primer 2: CDX2-p2) were chosen using the NEB LAMP Primer Design Tool (FIG. 3A). The CDX2-p1 primer set included the following primers: forward primer (F3) CGCCGAGCAGCTGTCT (SEQ ID NO: 1), backward primer (B3) GCTAGCTCGGCTTTCCTC (SEQ ID NO:2), forward internal primer (FIP) TTGGCTGCCGAGGGACTGCCAGCGGCGGAACCTGT (SEQ ID NO: 3), backward internal primer (BIP) TCGAGTGGTGTACACGGACCACGGATGGTGATGTAGCGACTG (SEQ ID NO: 4), loop primer forward (LF) GGCTTCCGCATCCACTC (SEQ ID NO: 5), loop primer backward (LB) CGGCTGGAGCTGGAGAA (SEQ ID NO: 6), forward internal primer (F2) CAGCGGCGGAACCTGT (SEQ ID NO: 7), forward internal primer (F1c) TTGGCTGCCGAGGGACTGC (SEQ ID NO: 8), backward internal primer (B2) GGATGGTGATGTAGCGACTG (SEQ ID NO: 9), and backward internal primer (B1c) TCGAGTGGTGTACACGGACCAC (SEQ ID NO: 10). The CDX2-p2 primer set included the following primers: forward primer (F3) GGTGTACACGGACCACCA (SEQ ID NO: 11), backward primer (B3) GCTGCTGCAACTTCTTCTTG (SEQ ID NO: 12), forward internal primer (FIP) CTCGGCTTTCCTCCGGATGGCTGGAGCTGGAGAAGGAGTT (SEQ ID NO: 13), backward internal primer (BIP) ACGCTGGGGCTCTCTGAGACTCTCCTTTGCTCTGCGG (SEQ ID NO: 14), loop primer backward (LB) GGCAGGTTAAAATCTGGTTTCA (SEQ ID NO: 15), forward internal primer (F2) CTGGAGCTGGAGAAGGAGTT (SEQ ID NO: 16), forward internal primer (F1c) CTCGGCTTTCCTCCGGATGG (SEQ ID NO: 17), backward internal primer (B2) CTCTCCTTTGCTCTGCGGTT (SEQ ID NO: 18), and backward internal primer (B1c) ACGCTGGGGCTCTCTGAGA (SEQ ID NO: 19).

For the human ACTB gene, two different exon boundaries were chosen (NM_001101.5:270-511, NM_001101.5:660-901) for the two primer sets respectively (Primer 1: ACTB-p1, Primer 2: ACTB-p2) (FIG. 3B). The ACTB-p1 primer set includes the following primers: forward primer (F3) AGTACCCCATCGAGCACG (SEQ ID NO: 20), backward primer (B3) AGCCTGGATAGCAACGTACA (SEQ ID NO: 21), forward internal primer (FIP) GAGCCACACGCAGCTCATTGTATCACCAACTGGGACGACA (SEQ ID NO: 22), backward internal primer (BIP) CTGAACCCCAAGGCCAACCGGCTGGGGTGTTGAAGGTC (SEQ ID NO: 23), loop primer forward (LF) TGTGGTGCCAGATTTTCTCCA (SEQ ID NO: 24), loop primer backward (LB) CGAGAAGATGACCCAGATCATGT (SEQ ID NO: 25), forward internal primer (F2) TCACCAACTGGGACGACA (SEQ ID NO: 26), forward internal primer (F1c) GAGCCACACGCAGCTCATTGTA (SEQ ID NO: 27), backward internal primer (B2) GCTGGGGTGTTGAAGGTC (SEQ ID NO: 28), and backward internal primer (B1c) CTGAACCCCAAGGCCAACCG (SEQ ID NO: 29). The ACTB-p2 primer set includes the following primers: forward primer (F3) GCTCGGCTACAGCTTCA (SEQ ID NO: 30), backward primer (B3) GGAAGAGTGCCTCAGGGC (SEQ ID NO: 31), forward internal primer (FIP) AAGTCCAGGGCGACGTAGCACCGGCCGGAGCGGGAAAT (SEQ ID NO: 32) backward internal primer (BIP) GAGATGGCCACGGCTGCTTCCATTGCCAATGGTGATGACCT (SEQ ID NO: 33), loop primer forward (LF) TTCTCCTTAATGTCACGCACG (SEQ ID NO: 34), loop primer backward (LB) CCCTGAGAAGAGCTACGAG (SEQ ID NO: 35), forward internal primer (F2) CGGCCGAGCGGGAAAT (SEQ ID NO: 36), forward internal primer (F1c) AAGTCCAGGGCGACGTAGCAC (SEQ ID NO: 37), backward internal primer (B2) TTGCCAATGGTGATGACCT (SEQ ID NO: 38), and backward internal primer (B1c) GAGATGGCCACGGCTGCTTCCA (SEQ ID NO: 39).

For the mouse Cdx2 gene, target region that includes two exon boundaries (NM_007673.3:713-1059) was used to search for the RT-LAMP primers (Primer 1: Cdx2-p1, Primer 2: Cdx2-p2) (FIG. 3C). The Cdx2-p1 primer set includes the following primers: forward primer (F3) AAACCTGTGCGAGTGGATG (SEQ ID NO: 40), backward primer (B3) TCGGAGAGCCCAAGTGTG (SEQ ID NO: 41), forward internal primer (FIP) GGTCTGTGTACACCACCCGGTAGCAGTCCCTAGGAAGCCA (SEQ ID NO: 42), backward internal primer (BIP) GGCTGGAGCTGGAGAAGGAGTGCCAGCTCACTTTTCCTCC (SEQ ID NO: 43), loop primer forward (LF) TGTCTTTTGTCCTGGTTTTCAC (SEQ ID NO: 44), loop primer backward (LB) CACTTTAGTCGATACATCACCATCA (SEQ ID NO: 45), forward internal primer (F2) GCAGTCCCTAGGAAGCCA (SEQ ID NO: 46), forward internal primer (F1c) GGTCTGTGTACACCACCCGGTA (SEQ ID NO: 47), backward internal primer (B2) GCCAGCTCACTTTTCCTCC (SEQ ID NO: 48), and backward internal primer (B1c) GGCTGGAGCTGGAGAAGGAGT) (SEQ ID NO: 49). The mouse Cdx2-p2 primers differ from the Cdx2-p1 primers only by the forward primer (F3) sequence, which is AGCGGCGAAACCTGTGCGAGT (SEQ ID NO: 50) for the Cdx2-p2 primer set.

For the mouse Actb gene, NM_007393.5:837-1238 target region was used to build two different primers (Primer 1: Actb-p1, Primer 2: Actb-p2) (FIG. 3D). The Actb-p1 primer set includes the following primers: forward primer (F3) ACCACCATGTACCCAGGC (SEQ ID NO: 51), backward primer (B3) GGCCGGACTCATCGTACT (SEQ ID NO: 52), forward internal primer (FIP) GCGCTCAGGAGGAGCAATGATCATTGCTGACAGGATGCAGAA (SEQ ID NO: 53), backward internal primer (BIP) TACTCTGTGTGGATCGGTGGCTTGCTTGCTGATCCACATCTG (SEQ ID NO: 54), loop primer forward (LF) TGGTGCTAGGAGCCAGAG (SEQ ID NO: 55), loop primer backward (LB) CCTCACTGTCCACCTTCCA (SEQ ID NO: 56), forward internal primer (F2) ATTGCTGACAGGATGCAGAA (SEQ ID NO: 57), forward internal primer (F1c) GCGCTCAGGAGGAGCAATCATC (SEQ ID NO: 58), backward internal primer (B2) TGCTTGCTGATCCACATCTG (SEQ ID NO: 59), and backward internal primer (B1c) TACTCTGTGTGGATCGGTGGCT (SEQ ID NO: 60). The Actb-p2 primer set includes the following primers: forward primer (F3) CCTGTGGCATCCATGAAACT (SEQ ID NO: 61), backward primer (B3) GGACAGTGAGGCCAGGAT (SEQ ID NO: 62), forward internal primer (FIP) CTGGGTACATGGTGGTACCACCAAGTGTGACGTTGACATCCG (SEQ ID NO: 63), backward internal primer (BIP) TACTGCTCTGGCTCCTAGCACCAGAGTACTTGCGCTCAGGA (SEQ ID NO: 64), loop primer forward (LF) TGTGTTGGCATAGAGGTCTTTA (SEQ ID NO: 65), loop primer backward (LB) TGAAGATCAAGATCATTGCTCC (SEQ ID NO: 66), forward internal primer (F2) AAGTGTGACGTTGACATCCG (SEQ ID NO: 67), forward internal primer (F1c) CTGGGTACATGGTGGTACCACC (SEQ ID NO: 68), backward internal primer (B2) AGAGTACTTGCGCTCAGGA (SEQ ID NO: 69), and backward internal primer (B1c) TACTGCTCTGGCTCCTAGCACC (SEQ ID NO: 70).

Each of these primers are referred to herein by name: CDX2-p1, CDX2-p2, ACTB-p1, ACTB-p2, Cdx2-p1, Cdx2-p2, Actb-p1, and Actb-p2.

### Example 4. Analysis of RT-PCR and RT-LAMP Data Using 5PL Model

Both RT-PCR and RT-LAMP requires primers to maintain specificity for a particular gene. RT-PCR uses two primers (F3, B3) (FIGS. 3A-3D) whereas RT-LAMP uses four to six primers (F3, B3, F1P=F1c::F2, BIP=B2::B1c, LB, LF) (FIGS. 3A-3D) for specific target amplification. The RT-PCR quantification is performed by measuring the amount of PCR product produced at each thermocycle step of the reaction or in “real time” by the quantity of the fluorescent signal. The point on the curve in which the amount of fluorescence begins to increase rapidly, usually a few standard deviations above the baseline, is termed the cycle threshold value (Ct value). Biologically, the higher the starting copy number of the nucleic acid target is, the sooner a significant increase in fluorescence is detected, and the lower the Ct value. In other words, the lower the Ct value, the higher the gene expression.

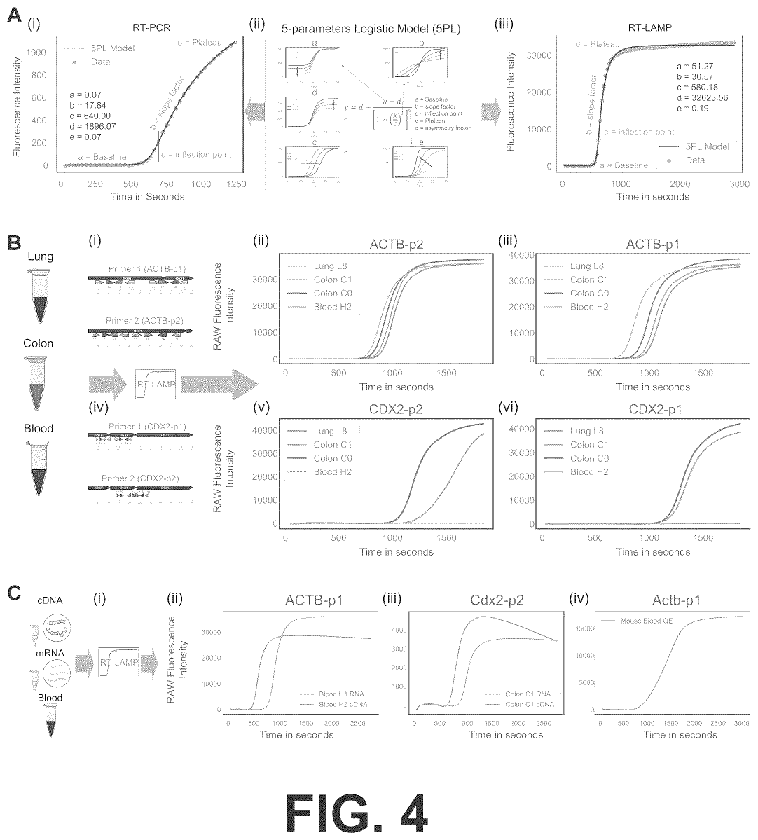

Both RT-PCR and RT-LAMP have a sigmoidal appearance when the fluorescence signal is plotted against time (in seconds) (FIG. 4A). These were modeled as logistic functions with five different parameters (5PL) (FIG. 4A (ii)): where a=Baseline, b=slope factor, c=inflection point, d=Plateau, e=asymmetry factor. These parameters are estimated using logistic regression (RT-PCR (FIG. 4A (i)), RT-LAMP (FIG. 4A (iii))). The inflection point can be used as Ct value for expression quantification. Baseline, Plateau and slope factor represent the starting value, saturation value and the rate of the amplification during exponential phase respectively. The changes in the shape of the curve by varying a single parameter is shown in FIG. 4A (ii).

### Example 5. RT-LAMP Amplified DNA Earlier than RT-PCR and to a Higher Level

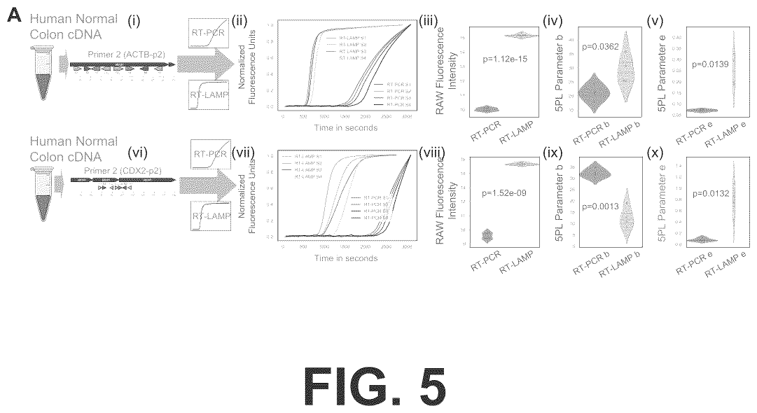

The RT-LAMP primer sets ACTB-p2 (FIG. 5A(i)) and CDX2-p2 (FIG. 5A(vi)) were tested in four different human colon cDNA samples. The fluorescence values were plotted against time in seconds in both RT-PCR and RT-LAMP data. For both primer sets ACTB-p2 (FIG. 5A(ii)) and CDX2-p2 (FIG. 5A(vii)), RT-LAMP amplified DNA earlier than RT-PCR (T-tests based on the inflection points: ACTB-p2, p=0.000132; CDX2-p2, p=0.000917). The saturation point for RT-LAMP was higher than RT-PCR for both ACTB-p2 (FIG. 5A(iii)) (p=1.1e−15) and CDX2-p2 (FIG. 5A(viii)) (p=1.5e−09). The slope factor for RT-LAMP was higher than RT-PCR in ACTB-p2 (FIG. 5A(iv)), whereas it was opposite in CDX2-p2 (FIG. 5A(ix)). However, the asymmetry factor of RT-LAMP was consistently higher than RT-PCR in both primer sets ACTB-p2 (FIG. 5A(v)) and CDX2-p2 (FIG. 5A(x)). The data agrees with previous findings that RT-LAMP increases the amount of amplified DNA even up to a billion copies over less than an hour, compared to a million copies yielded by the PCR^{1,2,14} Example 6. CDX2 RT-LAMP Expression is Specific to Colon Tissue

All four primers for the human gene ACTB and CDX2 were tested using RT-LAMP protocol (FIGS. 4A-4C) in four different human tissue cDNA samples (1× Lung, 2× Colon, 1× Blood) (FIGS. 4C(i), 4C(iv)) to check tissue specific expression patterns. As expected, cDNA was amplified in both ACTB-p2 (FIG. 4B(ii)) and ACTB-p1 (FIG. 4B(iii)) primer sets. CDX2 cDNA was amplified only in the two colon samples used for both CDX2-p2 (FIG. 4B(v)) and CDX2-p1 primer sets (FIG. 4B(vi)). The data agrees with previous findings that RT-LAMP is highly specific^{1,2,14}, and CDX2 is expressed specifically in the colon tissue¹⁵⁻¹⁷,

### Example 7. RT-LAMP can be Performed Directly from cDNA, mRNA and Tissue QuickExtract

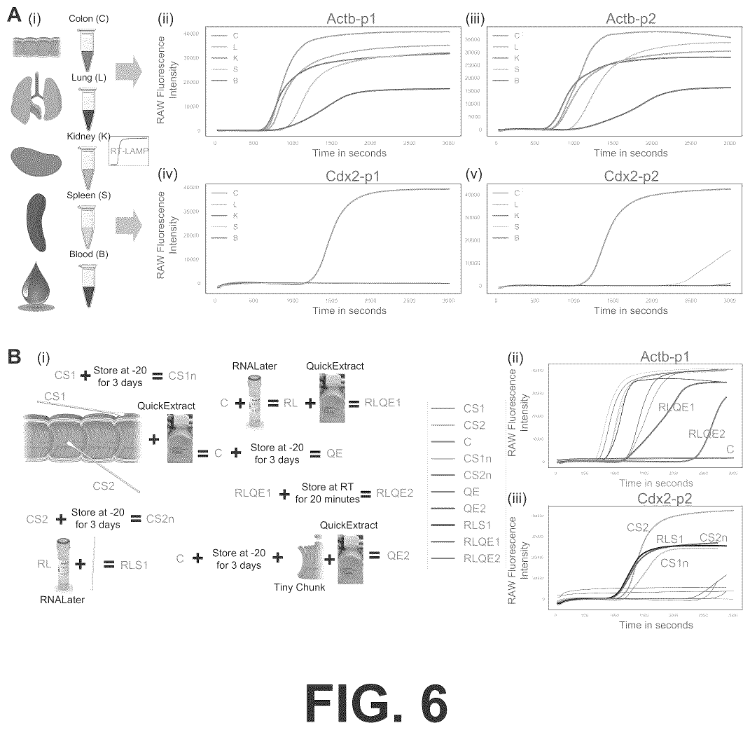

To test the versatility of RT-LAMP, three different sample types were used: cDNA, mRNA, and tissue QuickExtract. RT-LAMP using ACTB-p2 primer set was able to amplify DNA using both mRNA and cDNA samples from Blood (FIG. 4C(ii)). Unexpectedly, it was observed that CDX2 mRNA was being amplified from blood sample (Ct=35 minutes) (FIG. 4C(iii)) using RT-LAMP protocol, which was confirmed using GEL electrophoresis. CDX2 can be amplified using RT-PCR from blood sample based on previous reports¹⁸. These data suggest that both RT-PCR and RT-LAMP can amplify CDX2 mRNA from low amount. Next, whether gene expression can be measured directly from fresh tissue samples processed using Lucigen QuickExtract solution was tested. Mouse blood was processed using Lucigen QuickExtract solution and RT-LAMP protocol using Actb-p1 primer set was able to amplify Actb mRNA directly from the QuickExtract solution (FIG. 4C(iv)). To validate this further, mouse colon, lung, kidney, spleen, and blood samples were used to test on all four mouse primer sets (Actb-p1, Actb-p2, Cdx2-p1, Cdx2-p2) (FIG. 6A(i)). As expected, Actb-p1 (FIG. 6A(ii)) and Actb-p2 (FIG. 6A(iii)) amplified all samples, while only colon sample was amplified using Cdx2-p1 (FIG. 6A(iv)) and Cdx2-p2 (FIG. 6A(v)) primer set.

### Example 8. Cotton Swab on Colon Tissue Performs Better than Whole Chunk

Ten samples were subjected to RT-LAMP protocol for direct sample testing using Actb-p1 and Cdx2-p2 mouse primer set (FIG. 6B(i)). A cotton swab was used to lightly scratch the two different surfaces of the colon tissue (CS1 and CS2) and immerse them in Lucigen QuickExtract solution immediately. A whole chunk of the colon tissue was put in Lucigen QuickExtract solution (C) and stored the sample in the solution for 3 days in −20 C (QE). The cotton swab samples (CS1 and CS2) were also stored in −20 C for 3 days (CS1n and CS2n). A whole chunk of colon tissue was immersed in RNALater, stored in −20 C for three days, and later transferred to Lucigen QuickExtract solution (RLQE1) for one minute. RLQE1 is stored at room temperature for 20 minutes and processed later (RLQE2). The colon chunk stored in RNALater was scratched using a cotton swab and processed immediately using Lucigen QuickExtract solution (RLS1). A tiny piece of the whole colon chunk stored in Lucigen QuickExtract solution for 3 days in −20 C was transferred to a fresh Lucigen QuickExtract solution for one minute (QE2).

The ten samples were subjected to RT-LAMP protocol using Actb-p1 and Cdx2-p2 mouse primer set. The Actb-p1 primer set was able to amplify mRNA in almost all samples except the colon chunk in Lucigen QuickExtract (FIG. 6B(ii)). The colon chunk is probably too big to be processed using limited Lucigen QuickExtract solution. It was also observed that RLQE1 is amplified much earlier compared to RLQE2 (FIG. 6B(ii)). This suggest that mRNAs are being rapidly degraded ruling out inhibition of RT-LAMP activity by materials from tissue chunk in Lucigen QuickExtract solution. Accordingly, only cotton swab samples except CS1 (opposite side from the epithelium) were amplified using Cdx2-p2 primer set (FIG. 6B(iii)). Consistent with this pattern, cotton swab samples were amplified earlier using Actb-p1 RT-LAMP (FIG. 6B(ii)). Differential expression patterns between CS1 and CS2 suggest that Cdx2-p2 RT-LAMP is highly sensitive to colon epithelium. However, CS1 sample which was stored in room temperature for a while and −20 C for 3 days later called CS1n was amplified using Cdx2-p2 primer set (FIG. 6B(iii)). Collectively, these data suggest that cotton swab samples performed much better compared to whole colon chunks, and mRNAs are being degraded rapidly when whole colon tissue is processed using Lucigen QuickExtract solution.

### Example 9. RT-LAMP Based Gene Expression Matches Well with RT-PCR

To check the accuracy of gene expression measurement using RT-LAMP, two independent serial dilutions of human cDNA samples were prepared and processed using both RT-LAMP and RT-PCR protocols (FIG. 7A(i)). For RT-LAMP protocol CDX2-p2 and ACTB-p2 primer sets were used, while RT-PCR protocol used only the F3 and B3 primers from the CDX2-p2 and ACTB-p2 primer sets. Both RT-PCR and RT-LAMP data were modeled using five parameters logistic (5PL) function and all parameters were estimated using logistic regression. The inflection points for RT-PCR and RT-LAMP were highly correlated for both CDX2-p2 (r=0.99, p=1.27e−05) (FIG. 7A(ii)) and ACTB-p2 (r=0.97, p=0.000271) (FIG. 7A(ii)). Another independent serial dilution experiment showed similar results for both CDX2-p2 (r=0.97, p=4.03e−05) (FIG. 7A(iii)) and ACTB-p2 (r=0.98, p=1.94e−05) (FIG. 7A(iii)). This suggest that CDX2 expression can be reliably measured using RT-LAMP and faster than RT-PCR. These results may also be generalizable to fundamental nucleic acid detection using RT-LAMP techniques.

### Example 10. Modified RT-Lamp Primer Design Steps to Improve Quantification and Reduced False Positives

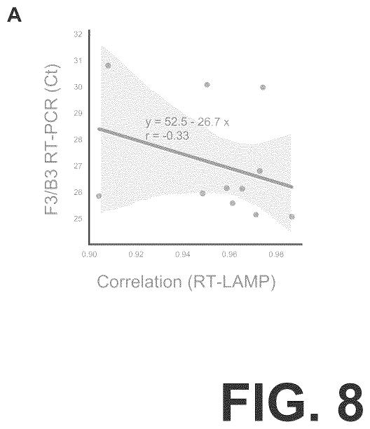

One of the major challenges for the RT-LAMP is the complex primer design steps which frequently produce primers that can self-amplify given enough time without any input sample. Self-amplification will lead to false positive in the result. Efficient quantification of RT-LAMP technologies depends on quick amplification of input nucleic acid and delayed or no self-amplification. A new relationship was discovered between efficient quantification and regular PCR amplification using F3/B3 part of the RT-LAMP primer (FIG. 8). This disclosure demonstrates that the stronger the PCR using F3/B3 the better the quantification of RT-LAMP (Ct=25, Correlation coefficient=0.99) in a serial dilution experiment. RT-LAMP primers can be screened for stronger F3/B3 PCR and weak/no self-amplification to identify the best reagents for mRNA quantification.

### Example 11. mRNA Expression can be Performed at Home

To demonstrate if mRNA expression measurement can be performed outside laboratory setting, a simple and affordable portable device was developed (length=4 in, width=3 in, height=5 in) that can be built using commercially available components (FIG. 7B). This work demonstrates that mRNA expression measurement will not be limited to laboratory setting only. Consumer access to this portable device, assay and data is possible which enables innovative applications in health care and hygiene. This will encourage students in elementary, middle, and high school to participate in the scientific discovery process and publish their results in scientific journals.

### 3D Design and Printing

3D design was carried out using Autodesk Tinkercad website and the designs were exported in STL format. The STL files were sliced using Ultimaker Cura 4.13.1 which generated gcode files. The gcode files were uploaded to 3D printers (Creality Ender 3 Pro 3D Printer with Removable Build Surface Plate and UL Certified Meanwell Power Supply Printing Size 8.66×8.66×9.84 in, Official Creality Ender 3 V2 3D Printer Upgraded Integrated Structure Design with Silent Motherboard Mean Well Power Supply and Carborundum Glass Platform 8.66×8.66×9.84 Inch, ANYCUBIC Mega S Upgrade FDM 3D Printer with Extruder and Suspended Filament Rack 8.27″ (L)×8. 27″ (W)×8.07″ (H) Print Size) using a microSD card. OVERTURE PLA Filament 1.75 mm PLA 3D Printer Filament, 1 kg Cardboard Spool (2.2 lbs), Dimensional Accuracy+/−0.03 mm (Royal Gold) was used for the printing process.

### PCB Design

MG Chemicals Copper Clad Board (Double Sided, 9″×6″, 1 oz Copper, 1/32″ Thick, FR4) was cut to desired shape using Dremel (8220-2/28 12-Volt Max Cordless Rotary Tool Kit with Battery) using Diamond Cutting Wheel (YEEZUGO ⅛″ Titanium Coating Diamond Cutting Discs Cut-Off Wheel Blades Shank Diameter 3.00 mm, Wheel diameter 1½″). The 2-sided copper clad board was cleaned using steel wool. A photosensitive dry film is thermally transferred to the copper board using GBC Thermal Laminator Machine (Fusion 7000L). PCB circuit is designed using Autodesk EAGLE software (version 9.6.2) and circuit mask is printed on transparency film using laser printer. The transparency films are attached to form an aligned two-sided circuit mask using scotch super hold transparent tape. Copper clad board with photoresist film is pushed between the two-sided circuit mask and exposed to strong UV light (Melody Susie 36W Nail Polish Curing Lamp and Sliding Tray) for five minutes. The UV exposed board is washed in soda ash (Sodium Carbonate, Na2CO3) solution and etched in Ferric Chloride for 1 hour. The etched board is washed in NaOH solution to expose the copper surface.

PCB is soldered using Aoyue 968A+ Professional SMD Digital Hot Air Rework Station with a Soldering Iron and Vacuum Pickup. Aluminum heating block is designed using six holes on a ⅜″ thick 6061-T651 Aluminum Plate Custom Cut: 1 in.×2 in. (± 1/16 in.). The holes are made using Drill Press (WEN 4214 12-Inch Variable Speed) and Step Drill Bit Set (NEIKO 10198A, 4-12 steps). PID heating controller (proportional with integral and derivative control) use MakerHawk NTC 3950 100K Thermistor and 12V 40W 620 Ceramic Cartridge Heater. Lee filters (777-rust and 071-Tokyo Blue, product ID 9409) were used to filter the fluorescence signal.

### REFERENCES

1. T. Notomi et al., Loop-mediated isothermal amplification of DNA. Nucleic Acids Res 28, E63 (2000).
2. M. Soroka, B. Wasowicz, A. Rymaszewska, Loop-Mediated Isothermal Amplification (LAMP): The Better Sibling of PCR? Cells 10, (2021).
3. A. Ganguli et al., Hands-free smartphone-based diagnostics for simultaneous detection of Zika, Chikungunya, and Dengue at point-of-care. Biomed Microdevices 19, 73 (2017).
4. A. Ganguli et al., Rapid isothermal amplification and portable detection system for SARS-COV-2. Proc Natl Acad Sci USA 117, 22727-22735 (2020).
5 A. Bektas et al., Accessible LAMP-Enabled Rapid Test (ALERT) for Detecting SARS-COV-2. Viruses 13, (2021).
6. G. L. Damhorst et al., Smartphone-Imaged HIV-1 Reverse-Transcription Loop-Mediated Isothermal Amplification (RT-LAMP) on a Chip from Whole Blood. Engineering (Beijing) 1, 324-335 (2015).
7 A. Ganguli et al., Pixelated spatial gene expression analysis from tissue. Nat Commun 9,202 (2018).
8. F. Wilisiani et al., Development of a LAMP assay with a portable device for real-time detection of begomoviruses under field conditions. J Virol Methods 265, 71-76 (2019).
9. D. A. Enicks, R. A. Bomberger, A. Amiri, Development of a Portable LAMP Assay for Detection of Neofabraea perennans in Commercial Apple Fruit. Plant Dis 104, 2346-2353 (2020).
10. G. Xun, S. T. Lane, V. A. Petrov, B. E. Pepa, H. Zhao, A rapid, accurate, scalable, and portable testing system for COVID-19 diagnosis. Nat Commun 12, 2905 (2021).
11. K. R. Sreejith et al., A Portable Device for LAMP Based Detection of SARS-COV-2. Micromachines (Basel) 12, (2021).
12. A. Rafiq et al., Development of a LAMP assay using a portable device for the real-time detection of cotton leaf curl disease in field conditions. Biol Methods Protoc 6, bpab010 (2021).
13. M. Inaba et al., Diagnostic accuracy of LAMP versus PCR over the course of SARS-CoV-2 infection. Int J Infect Dis 107, 195-200 (2021).
14. M. Parida, S. Sannarangaiah, P. K. Dash, P. V. Rao, K. Morita, Loop mediated isothermal amplification (LAMP): a new generation of innovative gene amplification technique; perspectives in clinical diagnosis of infectious diseases. Rev Med Virol 18, 407-421 (2008).
15. C. A. Moskaluk et al., Cdx2 protein expression in normal and malignant human tissues: an immunohistochemical survey using tissue microarrays. Mod Pathol 16, 913-919 (2003).
16. F. Drummond, W. Putt, M. Fox, Y. H. Edwards, Cloning and chromosome assignment of the human CDX2 gene. Ann Hum Genet 61, 393-400 (1997).
17. T. Mizoshita et al., Expression of Cdx1 and Cdx2 mRNAs and relevance of this expression to differentiation in human gastrointestinal mucosa—with special emphasis on participation in intestinal metaplasia of the human stomach. Gastric Cancer 4, 185-191 (2001).
18. R. El Omar et al., CDX2 regulates ACE expression in blood development and leukemia cells. Blood Adv 5, 2012-2016 (2021).

What is claimed is:

1. A method of detecting a nucleic acid in a biological specimen, the method comprising:
collecting a biological sample from the biological specimen;
preparing a test sample, the test sample comprising the biological sample and a plurality of nucleic acid primers specific for binding respective target nucleic acid sequences in the biological sample;
performing reverse transcription loop-mediated isothermal amplification (RT-LAMP) on the test sample; and
determining, from the RT-LAMP amplification, a quantity of the nucleic acid present in the biological sample.

2. The method of claim 1, wherein the biological specimen is selected from bacteria, viruses, archaea, fungi, biofilms, or combinations thereof.

3. The method of claim 1, wherein the biological specimen is selected from human tissue, saliva, blood, hair, urine, bodily secretions, or combinations thereof.

4. The method of claim 3, wherein the method is effective to diagnose a patient from which the biological sample is collected with gastrointestinal disease or colorectal cancer.

5. The method of claim 1, wherein the plurality of primers includes 4 to 6 primers selected from the group consisting of forward internal primers, backward internal primers, forward primers, backward primers, loop forward primers, loop backward primers, or combinations thereof.

6. The method of claim 5, wherein the target nucleic acid is CDX2, and wherein the plurality of primers are SEQ ID NOS: 1-10 or SEQ ID NOS: 11-19.

7. The method of claim 5, wherein the target nucleic acid is ACTB2, and wherein the plurality of primers are SEQ ID NOS: 20-29 or SEQ ID NOS: 30-39.

8. A method of identifying a plurality of primers for identifying a target nucleic acid in a biological sample using reverse transcription loop-mediated isothermal amplification (RT-LAMP), the method comprising:
providing a nucleotide sequence for the target nucleic acid to a computer program, wherein the computer program is configured to identify the plurality of primers based on a nucleotide sequence for the target nucleic acid.

9. The method of claim 8, wherein the plurality of primers are identified based on a concentration of nucleotide pairs, a location of nucleotide pairs, a distance between target DNA regions, self-amplification potential, PCR amplification time, or combinations thereof.

10. The method of claim 9, wherein the plurality of primers comprises 4-6 primers.

11. The method of claim 10, wherein the plurality of primers include at least one internal primer, at least one external primer, and/or at least one loop primer.

12. The method of claim 11, wherein the at least one internal primer is a forward internal primer or a backward internal primer, preferably wherein the forward internal primer is selected from the group consisting of F1c::F2, and wherein the backward internal primer is selected from the group consisting of B2::B2c.

13. The method of claim 12, wherein the at least one external primer is a forward primer or a backward primer.

14. The method of claim 13, wherein the forward primer is F3 and the backward primer is B3.

15. The method of claim 14, wherein the at least one loop primer is a loop forward primer or a loop backward primer, preferably wherein the loop forward primer is LF and the loop backward primer is LB.

16. The method of claim 8, wherein each of the plurality of primers is a single strand structure at a temperature from about 60° C. to about 65° C.

17. A desktop reverse transcription loop-mediated isothermal amplification (RT-LAMP) device comprising:
a housing defining a slot and a sample holder therein;
a display affixed to an exterior surface of the housing; and
a sample processing package disposed within the housing;
wherein the device is configured to detect a presence and quantity of a nucleic acid in a biological sample.

18. The desktop RT-LAMP device of claim 17, wherein the sample processing package comprises a filter, a camera, a heater, and a processor.

19. The desktop RT-LAMP device of claim 17, wherein the processor is configured to receive a user input, execute a command for performing RT-LAMP in accordance with the user input, process an output from the camera, and display the output on the display.

20. The desktop RT-LAMP device of claim 17, wherein the filter and the camera are positioned above the sample holder within the housing.

21. The desktop RT-LAMP device of claim 17, wherein the housing is less than 125 in³.

22. The desktop RT-LAMP device of claim 21, wherein the housing has a height of 5 in, a depth of 3 in, and a width of 4 in.

23. A method of using the desktop RT-LAMP device of any one of claim 17, the method comprising:
preparing a test sample comprising a biological sample and a plurality of primers;
providing the test sample to the sample holder within the housing;
supplying an input to the device; and
receiving an output via the display;
wherein the output received indicates the quantity of the nucleic acid present in the biological sample.

24. The method of claim 23, wherein the desktop RT-LAMP device is configured to detect colorectal cancer.
